(19) United States
(12) Patent Application Publication
(10) Pub. No.: US 2023/0301892 A1
(43) Pub. Date: Sep. 28, 2023

(54) COMPOSITIONS AND METHODS FOR ELONGATING CURLY HAIR

(71) Applicant: L'ORÉAL, Paris (FR)

(72) Inventors: Stephanie LAGA, Rahway, NJ (US); Rita CHOKSHI, Monroe Township, NJ (US); Heather LEE, Wayne, NJ (US)

(21) Appl. No.: 18/108,534

(22) Filed: Feb. 10, 2023

Related U.S. Application Data
(60) Provisional application No. 63/308,850, filed on Feb. 10, 2022.

(30) Foreign Application Priority Data
Apr. 21, 2022 (FR) 2203678

(51) Int. Cl. A61K 8/60; A61Q 5/06

(52) U.S. Cl. CPC A61K 8/60; A61Q 5/06; A61K 2800/48; A61K 2800/5922

(57) ABSTRACT

The disclosure relates to compositions comprising gluconolactone and at least one compound chosen from amino acids, amino sulfonic acids, and salts thereof. The compositions can be used for lengthening the visual appearance of hair, in particular curly hair. The disclosure also relates to methods of using the compositions.

### CROSS REFERENCE TO RELATED APPLICATIONS

This claims priority to U.S. Provisional Application No. 63/308,850, filed on Feb. 10, 2022, and French Application No. 2203678, filed on Apr. 21, 2022, both of which are incorporated by reference herein in their entireties.

### TECHNICAL FIELD

The present disclosure relates to compositions comprising gluconolactone and at least one compound chosen from amino acids, amino sulfonic acids, salts thereof, or mixtures thereof, and methods of using the compositions for styling hair such as improving elongation of curly hair.

### BACKGROUND

Curly hair usually appears shorter than its actual length. In the multicultural beauty market, there is a desire for products that can elongate hair curls and give more visual length to hair. In addition, reducing the curls of very curly hair may increase the manageability and ease of styling of such hair.

For curly hair consumers, points of concern include that their curly hair appears shorter than its actual length, and/or that the curls are not sufficiently defined. In the multicultural beauty market, there is a desire for products that elongate and/or define curls to showcase the natural curl pattern. Existing products on the market that aid in preventing curl shrinkage often use thick galenics with various butters or oils that can leave a greasy or oily feeling on hair. Products that provide hold, such as styling gels, can also give elongating effects, but these products typically flake on hair. Therefore, the use of these elongation products generally requires frequent hair washing, which is especially undesirable for consumers who have high curl patterns and prefer limited number of washdays per week and who desire to keep styles that last throughout the day or multiple days. In addition, traditional elongation of hair, including relaxing or straightening hair, often involves chemical treatment or high heat that may cause damage to the hair fibers and/or irritate the scalp.

As such, consumers desire new and improved styling compositions that can deliver visible and lasting elongation to hair curls and/or that provide visible and lasting curl definition and hold, particularly under humid conditions, and at the same time impart various additional advantageous properties to the hair such as moisture, softness, smoothness, good bounce, good shine, and/or frizz control.

It has now surprisingly been found that cosmetic compositions comprising gluconolactone and at least one compound chosen from amino acids, amino sulfonic acids, salts thereof, or mixtures thereof, are able to improve the elongation of hair curls and/or provide curl definition and hold, particularly under humid conditions, as well as provide other benefits such as hair moisture, softness, frizz control, and/or manageability benefits to the hair.

### SUMMARY

The present disclosure relates to compositions that have surprisingly been found to improve the elongation of hair curls. The compositions also provide curl definition and/or hold. The compositions are optionally leave-in hair styling compositions, and may, for example, be in the form of a serum, a gel, a gel cream, a cream, a lotion, or the like. The disclosure also relates to methods for styling hair, e.g. by improving the elongation of curly hair with the compositions.

The compositions comprise (a) gluconolactone, (b) at least one compound chosen from amino acids, amino sulfonic acids, salts thereof, or mixtures thereof, and (c) at least one solvent. In various embodiments, the pH of the composition is less than about 7. The compositions may optionally further comprise additional components such as, for example, polysaccharides, film forming polymers, anionic surfactants, thickening agents, buffers or pH adjustors, or mixtures thereof.

In various embodiments, the compositions comprise (a) from about 0.05% to about 10%, for example from about 0.1% to about 5%, from about 0.5% to about 3%, or from about 1% to about 2% of gluconolactone, (b) from about 0.05% to about 10%, for example from about 0.1% to about 8%, from about 0.5% to about 6%, from about 0.75% to about 4%, or from about 1% to about 3% of at least one compound chosen from amino acids, amino sulfonic acids, salts thereof, or mixtures thereof, and (c) at least one solvent, wherein all amounts are by weight, relative to the total weight of the composition.

In certain embodiments, the weight ratio of the total amounts of (b) amino acids + amino sulfonic acids + salts:(a) gluconolactone in the compositions ranges from about 5:1 to about 1:1, for example from about 4:1 to about 1:1, from about 3:1 to about 1:1, from about 2.5:1 to about 1:1, or from about 2:1 to about 1:1, such as about 2.5:1, about 2:1, about 1.5:1, or about 1:1.

In various embodiments, the pH of the composition is less than about 7, for example ranges from about 3 to about 6, from about 3.5 to about 5, from about 4 to about 4.5, or from about 4 to about 4.3. Optionally, the compositions comprise at least one buffer or pH adjustor.

In various embodiments, the compositions comprise at least one amino acid or amino sulfonic acid, optionally chosen from compounds of formulae (I)-(II) and/or salts thereof, for example chosen from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, minomethane sulfonic acid, aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, salts thereof, or mixtures thereof. In some embodiments, the compositions comprise, consist essentially of, or consist of glycine, taurine, phenylalanine, salts thereof, or mixtures thereof as the only amino acids/amino sulfonic acids. In further embodiments, the at least one amino acid/amino sulfonic acid comprises, consists essentially of, or consists of glycine.

In various embodiments, the compositions comprise at least one polysaccharide chosen from anionic polysaccharides, cationic polysaccharides, nonionic polysaccharides, or mixtures thereof. In some embodiments, the polysaccharides are chosen from inulins, celluloses, starches, and gums such as guar gums, xanthan gums, pullulan gums, alginate gums, agar-agar gums, carrageenan gums, gellan gums, gum arabics, xyloses, tragacanth gums, derivatives thereof, or mixtures thereof, for example chosen from hydroxypropyl guar, carrageenan, xanthan gum, cellulose gum, or mixtures thereof. In some embodiments, the polysaccharide is chosen from xanthan gum, carrageenan, hydroxypropyl guar, or mixtures thereof. In some embodiments, the polysaccharide comprises xanthan gum, hydroxypropyl guar, or a mixture thereof. In an embodiment, the polysaccharide comprises, consists essentially of, or consists of xanthan gum.

The compositions may optionally comprise at least one film forming polymer. In some embodiments, the film forming polymer comprises, consists essentially of, or consists of nonionic film forming polymer(s). The film forming polymer may, for example, be chosen from polyalkyloxazolines, vinyl acetate homopolymers, vinyl acetate copolymers, acrylic ester homopolymers and copolymers, copolymers of acrylonitrile and of a nonionic monomer, styrene homopolymers, styrene copolymers, copolymers of styrene and of butadiene, or copolymers of styrene, of butadiene and of vinylpyridine, polyamides, vinyllactam homopolymers, vinyllactam copolymers, vinylpyrrolidone/methacrylamide/vinylimidazole terpolymers, or mixtures thereof. In one embodiment, the composition comprises, consists essentially of, or consists of vinylpyrrolidone/methacrylamide/vinyl imidazole copolymer as the film forming polymer. In some embodiments, the compositions comprise a total amount of film forming polymers, for example nonionic film forming polymers such as vinylpyrrolidone/methacrylamide/vinyl imidazole copolymer, in an amount ranging from about 0.05% to about 2%, for example from about 0.1% to about 1%, or from about 0.2% to about 0.5% by weight, relative to the total weight of the composition.

In various embodiments, the compositions comprise at least one additional component, e.g. at least one anionic surfactant, at least one thickening agent, at least one pH adjustor, or mixtures thereof.

In further embodiments, the compositions comprise (a) from about 0.05% to about 10%, for example from about 0.1% to about 5%, from about 0.5% to about 3%, or from about 1% to about 2% of gluconolactone, (b) from about 0.05% to about 10%, for example from about 0.1% to about 8%, from about 0.5% to about 6%, from about 0.75% to about 4%, or from about 1% to about 3% of at least one amino acid or amino sulfonic acid, e.g. glycine, taurine, phenylalanine, salts thereof, or mixtures thereof, and (c) at least one solvent, wherein the weight ratio of (b) amino acids + amino sulfonic acids + salts:(a) gluconolactone ranges from about 5:1 to about 1:1, and all amounts are by weight, relative to the total weight of the composition.

In further embodiments, the compositions comprise (a) from about 0.05% to about 10%, for example from about 0.1% to about 5%, from about 0.5% to about 3%, or from about 1% to about 2% of gluconolactone, (b) from about 0.05% to about 10%, for example from about 0.1% to about 8%, from about 0.5% to about 6%, from about 0.75% to about 4%, or from about 1% to about 3% of at least one amino acid, amino sulfonic acid, salts thereof, or mixtures thereof, e.g. glycine, taurine, phenylalanine, salts thereof, or mixtures thereof, (c) at least one solvent, and (d) at least one additional component chosen from anionic surfactants, thickening agents, pH adjustors, or mixtures thereof, wherein the pH of the composition is less than about 7, wherein the weight ratio of (b) amino acids + amino sulfonic acids + salts:(a) gluconolactone ranges from about 5:1 to about 1:1, and wherein all amounts are by weight, relative to the total weight of the composition.

In further embodiments, the compositions comprise (a) from about 0.05% to about 10%, for example from about 0.1% to about 5%, from about 0.5% to about 3%, or from about 1% to about 2% of gluconolactone, (b) from about 0.05% to about 10%, for example from about 0.1% to about 8%, from about 0.5% to about 6%, from about 0.75% to about 4%, or from about 1% to about 3% of at least one amino acid, amino sulfonic acid, salts thereof, or mixtures thereof, e.g. glycine, taurine, phenylalanine, salts thereof, or mixtures thereof, (c) at least one solvent, and (d) optionally at least one additional component chosen from anionic surfactants, thickening agents, pH adjustors, or mixtures thereof, wherein the pH of the composition ranges from about 3 to about 6, for example from about 3.5 to about 5, from about 4 to about 4.5, or from about 4 to about 4.3; wherein the weight ratio of (b) amino acids + amino sulfonic acids + salts:(a) gluconolactone ranges from about 5:1 to about 1:1, for example from about 4:1 to about 1:1, from about 3:1 to about 1:1, from about 2.5:1 to about 1:1, or from about 2:1 to about 1:1; and wherein all amounts are by weight, relative to the total weight of the composition.

The disclosure further relates to methods of using the compositions for elongating curls, lengthening the visual appearance of curly hair, reducing shrinking of curly hair length, maintaining elongation of curls, and/or reducing frizz, even in conditions of high relative humidity, wherein the methods comprise applying the composition to wet, damp, or dry hair. Optionally, the methods do not include a heating step. The methods generally provide one or more styling benefits to the hair, such as curl elongation, curl definition, frizz control, smoothness, and/or softness.

### BRIEF DESCRIPTION OF THE FIGURES

The accompanying drawings, which are incorporated herein and constitute part of this specification, illustrate exemplary embodiments of the disclosure, and, together with the general description given above and the description provided herein, serve to explain features of the disclosure.

FIG. 1A is a graph comparing elongation (length retention) of hair swatches treated with compositions according to the disclosure and comparative compositions after exposure to high humidity.

FIG. 1B shows images of hair swatches treated with compositions according to the disclosure and comparative compositions at different time points.

FIG. 2 is a graph comparing elongation (length retention) of hair swatches treated with compositions according to the disclosure and a comparative composition at different time points.

FIG. 3A is a graph comparing elongation (length retention) of hair swatches treated with compositions according to the disclosure and comparative compositions after exposure to high humidity.

FIG. 3B shows images of hair swatches treated with compositions according to the disclosure and comparative compositions at different time points.

FIG. 4A is a graph comparing elongation (length retention) of hair swatches treated with a composition according to the disclosure and comparative compositions after exposure to high humidity.

FIG. 4B shows images of hair swatches treated with compositions according to the disclosure and a comparative composition at different time points.

It is to be understood that the foregoing and following descriptions are exemplary and explanatory only, and are not intended to be restrictive of any subject matter claimed.

### DETAILED DESCRIPTION

The disclosure relates to compositions for elongating curls, lengthening the visual appearance of curly hair, reducing shrinking of curly hair length, maintaining elongation of curls, and/or reducing frizz, even in conditions of high relative humidity, as well as to methods of using the compositions.

### I. Compositions

In various embodiments, compositions according to the disclosure comprise (a) gluconolactone; (b) at least one compound chosen from amino acids, amino sulfonic acids, salts thereof, or mixtures thereof; and (c) at least one solvent. Compositions according to the disclosure may improve hair curl elongation and/or persistence of hair curl elongation, and/or may provide additional benefits such as softness, smoothness, curl definition, curl hold, and/or frizz control to the hair.

### Gluconolactone

It has unexpectedly been discovered that gluconolactone has surprising hair styling benefits for lengthening the visual appearance of curly hair. Thus, compositions according to the disclosure comprise gluconolactone. The gluconolactone may be present in an amount ranging from about 0.05% to about 10% by weight, such as from about 0.05% to about 8% by weight, from about 0.05% to about 6% by weight, from about 0.05% to about 4% by weight, from about 0.05% to about 2.5%, from about 0.05% to about 2%, from about 0.05% to about 1.5%, from about 0.05% to about 1%, from about 0.1% to about 10%, from about 0.1% to about 8%, from about 0.1% to about 6%, from about 0.1% to about 4%, from about 0.1% to about 2.5%, from about 0.1% to about 2%, from about 0.1% to about 1.5%, from about 0.1% to about 1%, from about 0.5% to about 10%, from about 0.5% to about 8%, from about 0.5% to about 6%, from about 0.5% to about 4%, from about 0.5% to about 2.5%, from about 0.5% to about 2%, from about 0.5% to about 1.5%, from about 0.5% to about 1%, from about 1% to about 10%, from about 1% to about 8%, from about 1% to about 6%, from about 1% to about 4%, from about 1% to about 2.5%, or from about 1% to about 2% by weight, based on the total weight of the composition, including all ranges and subranges there between. In some embodiments, the compositions comprise gluconolactone in an amount ranging from about 0.1% to about 6%, for example from about 0.25% to about 5%, from about 0.5% to about 4%, from about 0.75% to about 3%, or from about 1% to about 2%, or may be about 0.5%, about 0.75%, about 1%, about 1.25%, about 1.5%, about 1.75%, or about 2% by weight, based on the total weight of the composition.

### Amino Acids / Amino Sulfonic Acids / Salts

It has also been surprisingly discovered that amino acids, amino sulfonic acids, and salts thereof work synergistically with gluconolactone to provide lengthening benefits to curly hair. Thus, compositions according to the disclosure additionally comprise at least one compound chosen from amino acids, amino sulfonic acids, salts thereof, or mixtures thereof. Amino acids are simple organic compounds containing both a carboxylic acid group (—COOH) and an amino group (—NH₂). Amino sulfonic acids are simple organic compounds containing both a sulfonic acid group (—SO₂OH) and an amino group (-NH₂). Accordingly, the amino acids and/or amino sulfonic acids may, in some embodiments, be chosen from compounds of formula (I), compounds of formula (II), salts thereof, or mixtures thereof:

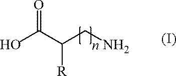

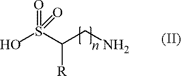

wherein:
R represents a hydrogen atom, a linear or branched, preferably linear, C₁-C₅ alkyl group, said alkyl group being optionally substituted with at least one group chosen from hydroxyl, —C(O)—OH, —S(O)₂—OH, —C(O)—O⁻ M⁺, and S(O)₂—O⁻ M⁺, where M⁺ represents a cationic counterion such as an alkali metal, alkaline earth metal, or ammonium, and
n is 0 or 1.

Useful amino acids that can be chosen include, but are not limited to, the twenty amino acids that form the proteins of living organisms (standard proteinogenic amino acids): alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine, and valine. In some embodiments, charged amino acids may be chosen. Non-limiting examples of charged amino acids include arginine, lysine, aspartic acid, and glutamic acid. In some embodiments, polar amino acids may be chosen. Non-limiting examples of polar amino acids include glutamine, asparagine, histidine, serine, threonine, tyrosine, cysteine, methionine, and tryptophan. In some embodiments, hydrophobic amino acids may be chosen. Non-limiting examples of hydrophobic amino acids include alanine, isoleucine, leucine, phenylalanine, valine, proline, and glycine.

Useful amino sulfonic acids that can be chosen include, but are not limited to, aminomethane sulfonic acid, aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, and amino hexyl benzene sulfonic acid.

Mixtures of two or more amino acids or salts thereof, such as three or more amino acids or salts thereof, may also be chosen. Mixtures of two or more amino sulfonic acids or salts thereof, such as three or more amino sulfonic acids or salts thereof, may also be chosen. Mixtures of one or more amino acids or salts thereof and one or more amino sulfonic acids or salts thereof may also be chosen.

In certain embodiments, the compositions comprise at least one compound chosen from glycine, alanine, serine, taurine, threonine, phenylalanine, salts thereof, or mixtures thereof, e.g. glycine, taurine, threonine, phenylalanine, salts thereof, or mixtures thereof. In further embodiments, the compositions comprise glycine and/or a salt thereof, either as the sole amino acid or in combination with one or more additional compounds chosen from amino acids and amino sulfonic acids. In yet further embodiments, the compositions comprise taurine and/or a salt thereof, either as the sole amino sulfonic acid or in combination with one or more additional compounds chosen from amino acids and amino sulfonic acids. In still further embodiments, the compositions comprise phenylalanine, glycine, taurine, salts thereof, or combinations thereof, optionally as the sole compounds chosen from amino acids, amino sulfonic acids, and their salts.

The total amount of compounds chosen from amino acids, amino sulfonic acids, or salts thereof in the composition may range from about 0.05% to about 10% by weight, such as from about 0.05% to about 8%, from about 0.05% to about 6%, from about 0.05% to about 4%, from about 0.05% to about 2.5%, from about 0.05% to about 2%, from about 0.05% to about 1.5%, from about 0.05% to about 1%, from about 0.1% to about 10%, from about 0.1% to about 8%, from about 0.1% to about 6%, from about 0.1% to about 4%, from about 0.1% to about 2.5%, from about 0.1% to about 2%, from about 0.1% to about 1.5%, from about 0.1% to about 1%, from about 0.5% to about 10%, from about 0.5% to about 8%, from about 0.5% to about 6%, from about 0.5% to about 4%, from about 0.5% to about 2.5%, from about 0.5% to about 2%, from about 0.5% to about 1.5%, from about 0.5% to about 1%, from about 1% to about 10%, from about 1% to about 8%, from about 1% to about 6%, from about 1% to about 4%, from about 1% to about 2.5%, or from about 1% to about 2% by weight, based on the total weight of the composition, including all ranges and subranges there between. In some embodiments, the compositions comprise a total amount of compounds chosen from amino acids, amino sulfonic acids, and/or salts thereof ranging from about 0.1% to about 7%, for example from about 0.25% to about 6%, from about 0.5% to about 5%, from about 0.75% to about 4%, or from about 1% to about 3%, or may be about 1%, about 1.25%, about 1.5%, about 1.75%, about 2%, about 2.25%, about 2.5%, about 2.75%, or about 3% by weight, based on the total weight of the composition.

In some exemplary embodiments, the compositions comprise from about 0.1% to about 5%, for example from about 0.5% to about 4%, or from about 1% to about 3% by weight of glycine, alanine, serine, taurine, threonine, phenylalanine, salts thereof, or mixtures thereof, relative to the total weight of the composition. In further exemplary embodiments, the compositions comprise from about 0.1% to about 5%, for example from about 0.5% to about 4%, from about 1% to about 3% by weight of glycine, taurine, salts thereof, or mixtures thereof, relative to the total weight of the composition.

In certain embodiments, the amounts of gluconolactone and compounds chosen from amino acids, amino sulfonic acids, and salts thereof are chosen so that the total amounts of (b) amino acids + amino sulfonic acids + salts and (a) gluconolactone provide optimal hair lengthening benefits. For example, the weight ratio of (b) amino acids + amino sulfonic acids + salts:(a) gluconolactone may range from about 5:1 to about 1:1, such as from about 4.5:1 to about 1:1, from about 4:1 to about 1:1, from about 3.5:1 to about 1:1, from about 3:1 to about 1:1, from about 2.5:1 to about 1:1, from about 2:1 to about 1:1, or from about 1.5:1 to about 1:1. In certain embodiments, the amounts of gluconolactone and compounds chosen from amino acids and amino sulfonic acids are chosen so that the weight ratio of the total amounts of (b) amino acids + amino sulfonic acids + salts:(a) gluconolactone is about 1:1, about 1.5:1, about 2:1, about 2.5:1, about 3:1, about 3.5:1, about 4:1, about 4.5:1, or about 5:1.

### Solvent

Compositions according to the disclosure comprise a cosmetically acceptable solvent. The solvent may comprise water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises one or more non-aqueous solvents, other than or in addition to ingredients discussed above. For example, C₁₋₄ alcohols, organic solvents, fatty alcohols, fatty ethers, fatty esters, polyols other than those described above, glycols, vegetable oils, mineral oils, liposomes, laminar lipid materials, or any a mixture thereof. Non-limiting examples of solvents which may be used include alkane polyols such as 1,2,6-hexanetriol, trimethylolpropane, ethylene glycol, propylene glycol, diethylene glycol, hexylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetine, triacetine, sulfolane, or mixtures thereof. At least in some embodiments, the compositions contain water and at least one, for example two or more, additional solvent chosen from caprylyl glycol, hexylene glycol, ethylhexylglycerin, glycerin, or mixtures thereof.

The solvent may be present in the composition in an amount ranging from about 50% to about 98% by weight, relative to the total weight of the composition, including all ranges and subranges therebetween. For example, in one embodiment, the total amount of solvent may be about 50% to about 95%, about 60% to about 95%, about 70% to about 95%, about 70% to about 90%, about 75% to 95%, or about 75% to 90% by weight, relative to the total weight of the composition. In certain embodiments, the solvent is primarily comprised of water, such as from about 90% to about 99%, or about 95% to about 99%, of the total solvent.

### Additional Components

Compositions according to the disclosure may optionally comprise one or more additional components suitable for use in such compositions. Non-limiting examples of such additional components are provided below.

### Film Forming Polymers

Compositions according to the disclosure optionally include one or more film forming polymers. In certain non-limiting embodiments, the film forming polymers are nonionic film forming polymers.

Useful and non-limiting examples of film forming polymers that may be chosen include polyalkyloxazolines; vinyl acetate homopolymers, vinyl acetate copolymers, such as, for example, copolymers of vinyl acetate and of acrylic ester, copolymers of vinyl acetate and of ethylene, or copolymers of vinyl acetate and of maleic ester, for example of dibutyl maleate; acrylic ester homopolymers and copolymers, such as, for example, copolymers of alkyl acrylates and of alkyl methacrylates, such as the products provided by Rohm GmbH under the name Eudragit® NE 30 D (INCI name: Acrylates copolymer); copolymers of acrylonitrile and of a nonionic monomer chosen, for example, from butadiene and alkyl (meth)acrylates; styrene homopolymers; styrene copolymers, such as, for example, copolymers of styrene, of alkyl acrylate and of alkyl methacrylate; copolymers of styrene and of butadiene; or copolymers of styrene, of butadiene and of vinylpyridine; polyamides; vinyllactam homopolymers, such as the vinylpyrrolidone homopolymers sold, for example, under the names Luviskol® K30 powder by the company BASF or PVP K30L or K60 solution or K90 by the company ISP, or such as the polyvinylcaprolactam sold under the name Luviskol® Plus by the company BASF (INCI name PVP); vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name VP/VA copolymer); and vinylpyrrolidone/methacrylamide/ vinylimidazole terpolymers, for instance the product sold under the name Luviset® Clear by the company BASF (INCI name VP/methacrylamide/vinyl imidazole copolymer). In various embodiments, the alkyl groups of these polymers optionally contain from 1 to 6 carbon atoms.

In certain embodiments, the film forming polymer is nonionic. In exemplary embodiments, the nonionic film forming polymer may be chosen from vinylpyrrolidone/methacrylamide/vinylimidazole terpolymers. In some embodiments, the compositions comprise vinylpyrrolidone/methacrylamide/vinyl imidazole copolymer, either as the sole film forming polymer or in combination with one or more additional film forming polymers.

If present, the total amount of film forming polymer may range from about 0.05% to about 10% by weight, such as from about 0.05% to about 8%, about 0.05% to about 6%, about 0.05% to about 5%, about 0.05% to about 4%, about 0.05% to about 3%, about 0.05% to about 2%, about 0.05% to about 1%, about 0.1% to about 10%, about 0.1% to about 8%, about 0.1% to about 6%, about 0.1% to about 5%, about 0.1% to about 4%, about 0.1% to about 3%, about 0.1% to about 2%, or about 0.1% to about 1% by weight, relative to the total weight of the composition.

In exemplary embodiments, the compositions comprise from about 0.05% to about 2%, for example from about 0.1% to about 1%, from about 0.2% to about 0.5% by weight of nonionic film forming polymers, for example vinylpyrrolidone/methacrylamide/vinyl imidazole copolymer, relative to the total weight of the composition.

### Anionic Surfactants

Compositions according to the disclosure optionally comprise at least one anionic surfactant. In various embodiments, suitable anionic surfactants may be chosen from sulfate/sulfonate, non-sulfate, or carboxylate anionic surfactants. For example alkyl sulfates, alkyl ether sulfates, alkyl monoglyceride sulfates, alkylaryl sulfates, alkyl ether carboxylates, fatty acyl sarcosinates, fatty acyl glutamates, alkyl phosphates, alkyl sulfoacetates, alkoxylated monoacids, alkyl sulfonates, alkyl amidesulfonates, alkylaryl sulfonates, alkyl monoglyceride sulfonates, α-olefin sulfonates, paraffin sulfonates, alkyl sulfosuccinates, alkyl ether sulfosuccinates, alkyl amidesulfosuccinates, alkyl sulfoacetates, N-acyltaurates, acylisethionates, alkyl sulfolaurates, salts thereof, or mixtures thereof may be chosen, wherein the alkyl groups of these compounds optionally including from 6 to 30 carbon atoms, such as from 12 to 28 carbon atoms, from 14 to 24 carbon atoms, or from 16 to 22 carbon atoms, wherein the aryl group of these compounds is optionally chosen from phenyl or benzyl groups, and wherein these compounds are optionally polyoxyalkylenated, for example polyoxyethylenated including from 1 to 50 ethylene oxide units such as from 2 to 10 ethylene oxide units. In certain embodiments, the anionic surfactant comprises one or more alkyl sulfonates.

When the anionic surfactants are in salt form, they may be chosen from, for example, alkali metal salts such as the sodium or potassium salts, ammonium salts, amine salts such as the amino alcohol salts, or alkaline-earth metal salts such as the magnesium salt.

In various embodiments, the anionic surfactants may be chosen from, for example, C6-C24 alkyl sulfates, for example C12-C20 alkyl sulfates; C6-C24 alkyl ether sulfates, for example C12-C20 alkyl ether sulfates, for example comprising from 2 to 20 ethylene oxide units; C6-C24 alkyl sulfosuccinates, for example C12-C20 alkyl sulfosuccinates, e.g. lauryl sulfosuccinates; C6-C24 alkyl ether sulfosuccinates, for example C12-C20 alkyl ether sulfosuccinates; C6-C24 acylisethionates, for example C12-C18 acylisethionates; C6-C24 acylsarcosinates, for example C12-C20 acylsarcosinates, e.g. palmitoylsarcosinates; C6-C24 alkyl ether carboxylates, for example C12-C20 alkyl ether carboxylates; polyoxyalkylenated (C6-C24) alkyl(amido) ether carboxylic acids and salts thereof, e.g. those having from 2 to 50 alkylene oxide, and in particular ethylene oxide, groups; C6-C24 acylglutamates, for example C12-C20 acylglutamates; C6-C24 acylglycinates, for example C12-C20 acylglycinates; or mixtures thereof.

In at least certain embodiments, the compositions may comprise at least one anionic surfactant chosen from linear α-olefin sulfonates or salts thereof. For example, the compositions may comprise at least one anionic surfactant chosen from compounds of formula (III) or salts thereof:

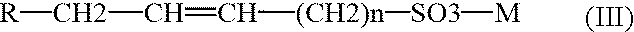

wherein:
R is a saturated linear alkyl radical comprising from 4 to 30 carbon atoms, such as from 6 to 20 carbon atoms or from 8 to 18 carbon atoms, and for example from 10 to 14 carbon atoms;
n is an integer ranging from 0 to 10, such as from 1 and 4, and preferably n=1; and
M is a cosmetically acceptable cation, for example cations derived from alkali metals or alkaline-earth metals or cations derived from organic amines such as alkanolamines, and preferably is chosen from ammonium, sodium, or potassium.

In certain embodiments, the at least one anionic surfactant is chosen from linear α-olefin sulfonates according to formula (IV) or salts thereof:

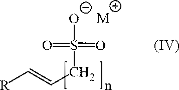

wherein R is an alkyl group, M is a sodium cation, and n is 1 or 2. For example, the at least one anionic surfactant may be sodium C14-C16 olefin sulfonate.

In various embodiments, if present, the total amount of anionic surfactants may range up to about 10% by weight, based on the total weight of the composition. For instance, the total amount of the at least one anionic surfactant may range from about 0.01% to about 10%, from about 0.01% to about 8%, from about 0.01% to about 5%, from about 0.01% to about 3%, from about 0.01% to about 2%, from about 0.01% to about 1%, from about 0.05% to about 10%, from about 0.05% to about 8%, from about 0.05% to about 5%, from about 0.05% to about 3%, from about 0.05% to about 2%, from about 0.05% to about 1%, from about 0.1% to about 10%, from about 0.1% to about 8%, from about 0.1% to about 5%, from about 0.1% to about 3%, from about 0.1% to about 2%, or from about 0.1% to about 1% by weight, relative to the total weight of the composition, including all ranges and subranges therebetween.

In certain embodiments, the compositions comprise at least one anionic surfactant which may be chosen from anionic sulfate or sulfonate surfactants, e.g. alkyl sulfonate surfactants and/or linear α-olefin sulfonates. In various embodiments, the anionic sulfate or sulfonate surfactants such as alkyl sulfonate surfactants and/or linear α-olefin sulfonates may be present in an amount ranging from about 0.01% to about 2.5% by weight, for example from about 0.1% to about 1% by weight, based on the total weight of the composition.

### Thickening Agents

Compositions according to the disclosure optionally include one or more thickening agents, also referred to as rheology modifying agents. The type and amount of thickening agent(s), if present, will be chosen depending on the form of the composition and desired viscosity thereof. Exemplary and non-limiting thickening agents include, for example, polysaccharides. For example, the polysaccharides may be chosen from anionic polysaccharides, cationic polysaccharides, nonionic polysaccharides, or mixtures thereof, such as anionic polysaccharides, cationic polysaccharides, or mixtures thereof. In various embodiments, the thickening agents may be chosen from celluloses, starches, guar gums, xanthan gums, pullulan gums, alginate gums, agar-agar gums, carrageenan gums, gellan gums, gum arabics, xyloses, tragacanth gums, or derivatives of the foregoing.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosaccharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxymethylcellulose, methylhydroxyethylcellulose, hydroxyethylcelluloses, hydroxypropylmethylcellulose, hydroxybutylcellulose, hydroxyethylmethylcellulose (also known as methyl hydroxyethylcellulose) or hydropropylcelluloses, which may or may not contain a fatty chain. Celluloses modified with groups comprising one or more nonionic fatty chains that can be used include hydroxyethylcelluloses, e.g. nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose. Other particular thickening agents suitable for use in the compositions described herein are hydroxypropyl guar gums.

Non-limiting examples of starches include modified starches, starch-based polymers, methylhydroxypropyl starch, potato starch, modified potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the thickening agents are chosen from carrageenan, xanthan gum, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropylguar gum, hydroxyethyl guar gum, hydroxypropyl guar, xylose gum, cellobiose, dextrin, maltodextrin, scleroglucan, chitosan, ulvan, fucoidan, alginate, pectin, heparin, inulin, levan, or mixtures thereof. In further embodiments, the thickening agents are chosen from sclerotium gum, carrageenan, xanthan gum, cellulose gum, cyclodextrin, or mixtures thereof.

In various exemplary embodiments, the total amount of thickening agents may range from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, or from about 1% to about 2% by weight, relative to the total weight of the composition, including all ranges and subranges thereof.

### Auxiliary Components

Useful auxiliary components that may be chosen include, but are not limited to, conditioning agents, dyes/pigments, moisturizing agents, fillers, structuring agents, shine agents, antioxidants, penetrants, sequestrants, fragrances, buffers (e.g. monoethanolamine and/or triethanolamine), dispersants, plant extracts, plant oils, preserving agents (preservatives), opacifiers, sunscreen agents, pH adjusting agents (e.g. citric acid), vitamins (e.g. Vitamin E), film forming polymers other than nonionic film forming polymers, surfactants other than anionic surfactants, and antistatic agents.

Optional additional auxiliary components may be present in an amount ranging up to about 15% by weight, such as from about 0.01% to about 15% or about 0.1% to about 10% by weight, relative to the total weight of the composition.

In various embodiments, the compositions may have a pH less than or equal to about 7. For example, the pH of the compositions may range from about 3 to about 6, such as from about 3.5 to about 5, from about 4 to about 4.5, or from about 4 to about 4.3, including all ranges and subranges therebetween. In various embodiments, the compositions comprise a buffering agent or pH adjustor in order to achieve the desired pH value.

Compositions according to the disclosure are typically in the form of a liquid, cream, gel, gel-cream, serum, or lotion, but are not limited to these forms. In an exemplary embodiment, the composition is a serum.

### II. Methods

The present disclosure also relates to methods for styling hair, e.g. by improving elongation and/or visual length of curly hair with the compositions described herein. The methods generally comprise applying any of the compositions according to the disclosure to the hair. The lengthening effects provided have surprisingly been demonstrated to last over time, even under conditions of high humidity.

Compositions according to the disclosure are typically used for providing a visible elongation benefit to wavy or curly hair, for improving the elongation of hair waves or curls, for providing long lasting elongation of hair waves or curls (even at high humidity conditions and/or over a period of time), for improving curl definition, for providing long lasting curl definition of wavy or curly hair, and/or for reducing frizziness of hair. The compositions may be useful in a variety of settings, either for chemically treated or untreated hair, and/or for natural or unnatural (e.g. permed hair) waves or curls. The compositions described herein are particularly suited to multicultural hair types of varying curliness and hair types. The degree of curliness or curl type of the hair may vary and is not limited. For example, the curls of hair been treated by the compositions disclosed herein may range from slightly wavy to very kinky and coily hair, and may have different textures and colors.

In some embodiments, the compositions described herein are useful for increasing the perceived length of hair, reducing shrinkage of curly hair, and/or reducing frizziness of hair, particularly in humid and high humidity (e.g. 80% RH, 25° C.) conditions, by providing lasting curl elongation and/or curl definition of the hair, e.g. providing lasting curl elongation and/or curl definition for 1 hour or more, for 4 hours or more, for 8 hours or more, for 12 hours or more, for 16 hours or more, for 24 hours or more, or for 72 hours or more, after application of a composition according to the disclosure.

In other embodiments, the compositions are useful for improving definition of curls and/or curl hold, and/or reducing frizziness of hair, particularly in humid and high humidity (e.g. 80% RH, 25° C.) conditions, by providing lasting curl elongation and/or curl definition of the hair, e.g. providing lasting curl elongation and/or curl definition for 1 hour or more, for 4 hours or more, for 8 hours or more, for 12 hours or more, for 16 hours or more, for 24 hours or more, or for 72 hours or more, after application of a composition according to the disclosure.

In addition, the compositions may also be used to provide a variety of desirable sensory benefits, for example, moisture, smoothness, softness, good bounce, and/or good shine, to the hair. As such, the compositions are useful in styling (which includes shaping) hair while also caring for hair, conditioning hair, and/or imparting one or more above described sensory benefits to the hair.

Typically, methods according to the disclosure may include applying an effective amount of a composition disclosed herein to hair having curls, when the hair is dry, wet, damp, or moist. As used herein, the term “effective amount” refers to an amount sufficient to provide a desired elongation effect to the hair, taking into account the degree of curliness, the length, the volume, and the texture of the hair. In general, from about 0.5 grams to about 50 grams of product is applied to the hair, depending on the specific product formulation, hair length, hair volume, and hair style type. The composition applied to the hair may be distributed evenly by combing through with fingers or a means such as a comb or the like. The composition may then be dried, for example air-dried. The composition may be allowed to remain on the hair as a leave-in product for any period of time as needed, for example, from about a few seconds (e.g. 1, 3, 5, or 10 seconds) to about 10, 20, or 30 minutes, or longer, such as a few hours or a few days, or until the next washing or rinsing of the hair.

In some embodiments, before applying the composition to the hair, the hair may be first cleansed with a commercially available shampoo, or rinsed with water. The composition is then applied to the washed or rinsed hair when the hair is wet, damp, or moist. In some other embodiments, before applying the composition to dry hair, the hair can optionally be moistened, damped, or wetted by water spray or using a wet towel, or by applying other treatment compositions that make the hair moist, damp, or wet.

Alternatively, in some embodiments, the compositions according to the disclosure may be first applied to dry hair, and then water or a cosmetically acceptable compositions are applied to the hair to moist, damp, or wet the hair when the compositions according to the disclosure are remained thereon. The hair that has been applied with the compositions disclose herein may be air-dried without further treatment of the hair, including rinsing or washing the hair.

In some embodiments, the methods may optionally include braiding and/or twisting out the hair to make curls, before or after application of the compositions disclosed herein. In some embodiments, methods according to the disclosure do not require shaping or styling the hair before, during, and/or after applying the compositions. A tension to the hair is generally not needed. As such, in some embodiments, before, during, or after applying the compositions, the hair is not stretched by a mechanical means.

In some embodiments, after application of the compositions disclosed herein, the hair may be shaped or styled as needed, such as be twisted or the like. In some embodiments, the hair treated with the compositions may be twisted within 30 minutes, or within 20 minutes, while the hair is wet or damp.

Methods according to the disclosure generally do not require the use of a reducing agent, including a base, or heating the hair for stretching the hair curls or elongating the hair curls. As such, in at least some embodiments, the method does not including using a reducing agent, or a base, and/or does not require or include a step of heating the hair after the composition is applied thereto in order to achieve the desired elongation results, although these may optionally be used if desired.

In some embodiments, a composition disclosed herein is used alone to treat the hair, without the use of other hair treatment compositions. In some other embodiments, the compositions may be applied to the hair before, during, or after another hair treatment or styling composition, such as (e.g. a conditioner, a mask, a cream, a lotion, a gel, a coloring composition, etc.), is applied to the hair. As such, a composition disclosed herein may be applied to the hair and layered with another composition that is also applied to the hair.

In various embodiments, the methods of treating hair with the compositions according to the disclosure, impart various long-lasting benefits described above to the hair, without a greasy feel or flaking, relative to hair not having been treated with a composition according to the disclosure. The term “long-lasting,” as used herein, means that the elongation effect and other benefits imparted to the hair may remain a desired length of time, such as a few hours, a few days, or until the hair is rinsed or washed.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/- 10% from the stated number, such as +/-9%, +/- 8%, +/- 7%, +/- 6%, +/- 5%, +/- 4%, +/- 3%, +/- 2%, or +/- 1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the term “applying a composition onto hair” and variations thereof means contacting the hair with at least one of the compositions of the disclosure, in any manner. It may also mean contacting the hair in an effective amount.

As used herein, the term “styling” the hair is intended to include “shaping,” the hair or altering the shape of hair, such as, for example, elongating hair curls or providing curl definition.

As used herein, the term “curly hair” refers to any hair including a curl or wave. The curl may be natural or unnatural, i.e., formed by chemical treatment or physical treatment of the hair. The degree of curliness of the hair may vary and is not limited.

As used herein, hair with improved or enhanced curl definition may have curls with a shape that has a clean ringlet appearance rather than being frizzy, curls that appear more individualized, curls that are more closed in appearance, and/or curls that have an improved visual appearance of the hair color and/or highlights.

As used herein, the terms “elongating” and “stretching” hair are interchangeable, and both refer to reducing the degree of waviness or curliness and/or tightness of waves or curls of the hair, and “improving the elongation of” hair or hair curls, and variants thereof, means that the length of the hair appears closer to the actual, uncurled length of the hair. Similarly, measuring or evaluating the “elongation” of curly hair refers to the length of hair retained over a period of time, where hair having a greater elongation or length retention correspondingly has a longer appearance over such time period.

A “leave-in” composition or product refers to a composition such as a hair-treatment or hair-styling composition that is not rinsed and/or washed away with water or acceptable solvent after the application of the composition onto the hair; instead, the composition is allowed to remain on the hair for a period of time as desired, such from about 1 hour, 2 hours, 3 hours, 4 hours, up to 8 hours, overnight, or as long as needed, until next time of washing or rinsing the hair.

As used herein, the term “inorganic” means a material that does not comprise carbon.

As used herein, the term “high humidity” refers to conditions having at least 70% relative humidity or greater, such as at least 75% relative humidity or greater, or at least 80% relative humidity or greater.

As used herein, the term “organic” means a material that is produced substantially without or essentially without the use of synthetic materials. “Organic materials” include materials made of organic compounds/molecules comprising at least one carbon atom.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” throughout the disclosure may include salts having a counter-ion such as an alkali metal, alkaline earth metal, or ammonium counterion. This list of counterions, however, is non-limiting. It should be understood that salts include salts that form in the composition unless otherwise indicated.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt%) of active materials, relative to the total weight of the composition.

### Example 1-1 — Compositions With Glycine and Various Concentrations of Gluconolactone

Compositions 1A-1D in Table 1 were prepared by combining glycine with varying amounts of gluconolactone in water, and the pH adjusted with a small amount of either citric acid or trisodium citrate as needed to a pH of about 4.1. A first control (C1) comprised only water, and a second control (C2) comprised only water with a small amount of trisodium citrate.

TABLE 1

| | Control | | Comparative | | | Inventive | | | |
|---|---|---|---|---|---|---|---|---|---|
| | C1 | C2 | C3 | C4 | C5 | 1A | 1B | 1C | 1D |
| Glycine | | | 2 | | | 2 | 2 | 2 | 2 |
| Gluconolactone | | | | 1 | 5 | 1 | 2 | 3 | 5 |
| Water | 100 | QS to 100 | QS to 100 | QS to 100 | QS to 100 | QS to 100 | QS to 100 | QS to 100 | QS to 100 |
| Ratio of Glycine: Gluconolactone | - | - | - | - | - | 2:1 | 1:1 | 0.67:1 | 0.4:1 |

Identical swatches of very curly hair were washed with a standard shampoo, rinsed, and detangled (Time -1). The swatches were dipped in one of compositions 1A-1D or C1-C5 and excess water was removed by squeezing by passing each swatch through the fingers five (5) times, which also served to help mechanically stretch the hair (Time 0). The treated hair swatches were allowed to air dry for 18 hours (Time 1). The treated swatches were then put into a humidity chamber at 80% relative humidity at 25° C. for 8 hours (Time 2). The hair swatches treated with each of compositions 1A-1D and C1 were evaluated for elongation (length retention), curl definition, and frizz at each of Time 0, Time 1, and Time 2.

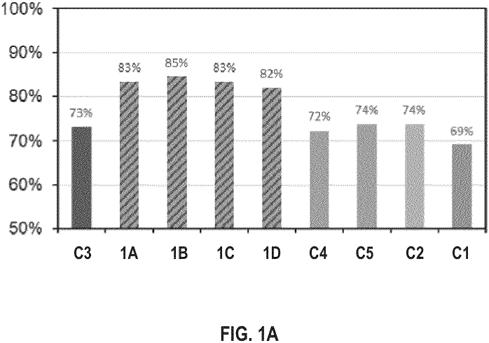

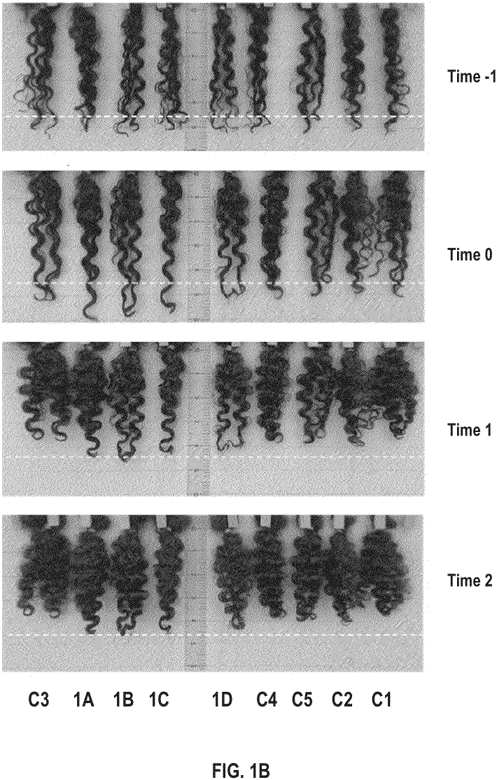

FIG. 1A shows that, at Time 2, each of compositions 1A-1D containing glycine and varying amounts of gluconolactone surprisingly provided significant improvement in length retention to very curly hair, compared to that provided by compositions C1-C5, even after high humidity. FIG. 1B shows that each of the swatches treated with compositions 1A-1D demonstrated markedly better elongation, curl definition, and frizz control than the swatches treated with compositions C1-C5 at each time point, even when subjected to high humidity.

Example 1-1 thus demonstrates that glycine in combination with gluconolactone in various concentrations and ratios surprisingly provides synergistic beneficial results to curly hair, such as elongation, curl definition, and frizz control.

### Example 1-2 — Compositions With Glycine and Various Concentrations of Gluconolactone

The study in Example 1-1 was repeated a second time on a second set of very curly hair swatches, with similar results wherein compositions 1A-1D provided better elongation, curl definition, and frizz control than compositions C1-C5, even when the hair was subjected to high humidity.

Example 1-2 therefore confirms the results of Example 1-1 that glycine in combination with gluconolactone in various concentrations and ratios surprisingly provides synergistic beneficial results to curly hair, such as elongation, curl definition, and frizz control.

### Example 2-1 — Compositions With Glycine and Gluconolactone at Varying pH

Inventive compositions 2A-2D in Table 2A were prepared by combining identical amounts of gluconolactone and glycine with water in the amounts shown, and adding a pH adjustor (citric acid or trisodium citrate) in a quantity sufficient to achieve the final specified pH. A control (C1) comprised only water.

TABLE 2A

| | Control | Inventive | | | |
|---|---|---|---|---|---|
| | C1 | 2A | 2B | 2C | 2D |
| Gluconolactone | | 1 | 1 | 2 | 2 |
| Glycine | | 2 | 2 | 2 | 2 |
| Citric acid or trisodium citrate | | QS | QS | QS | QS |
| Water | 100 | QS to 100 | QS to 100 | QS to 100 | QS to 100 |
| Measured pH value | - | 3.3 | 4.1 | 5.0 | 6.0 |

Identical swatches of very curly hair were washed with a standard shampoo, rinsed, and detangled. Equal amounts of inventive compositions 2A-2D or the control C1 were applied to separate wet hair swatches and distributed by raking through the curls to ensure even distribution (Time 0). The treated hair swatches were then allowed to air dry for 18 hours, at which time the film on the hair was broken by running fingers through each swatch from top to bottom three (3) times (Time 1). The treated swatches were then put into a humidity chamber at 80% relative humidity at 25° C. for 8 hours (Time 2).

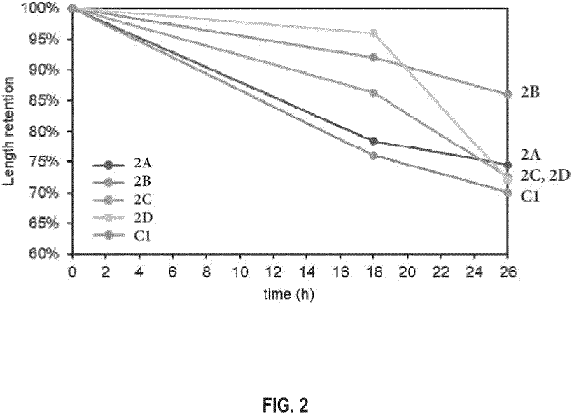

The hair swatches treated with one of compositions 2A-2D or the control C1 were evaluated for length at Time 1 compared to length at Time 0, and for length at Time 2 compared to length at Time 1, to determine the length retention of each swatch over the relevant time period. FIG. 2 shows that each of compositions 2A-2D provided improvement in length retention to very curly hair, compared to the control C1. The results are also shown in Table 2B.

TABLE 2B

| Length Retention Over Time | Control | Inventive | | | |
|---|---|---|---|---|---|
| | C1 | 2A | 2B | 2C | 2D |
| Time 0 → Time 1 | 76% | 78% | 92% | 86% | 96% |
| Time 1 → Time 2 | 70% | 75% | 86% | 73% | 72% |

Example 2-1 demonstrates that compositions having gluconolactone and glycine provide beneficial length retention to curly hair across a range of pH values.

### Example 2-2 — Compositions With Glycine and Gluconolactone at Varying pH

The study in Example 2-1 was repeated a second time on a second set of very curly hair swatches, with similar results wherein compositions 2A-2D provided better length retention than C1, even when the hair was subjected to high humidity.

Example 2-2 therefore confirms the results of Example 2-1 that glycine in combination with gluconolactone at various pH values provides synergistic beneficial length retention to curly hair.

### Example 3-1 - Hair Styling Compositions

The inventive and comparative hair styling compositions in Table 3 were prepared by combining the components shown with water and a pH adjustor in a quantity sufficient to achieve a final pH in the range of about 4.2 to about 5.2.

TABLE 3

| | Inventive | | Comparative | | | | | |
|---|---|---|---|---|---|---|---|---|
| | 3A | 3B | C6 | C7 | C8 | C9 | C10 | C11 |
| Glycine | 2 | 2 | 2 | | | 2 | | |
| Gluconolactone | 1 | 1 | | 1 | | | 1 | |
| Xanthan gum | 0.75 | | | | 0.75 | 0.75 | 0.75 | |
| Phenoxyethanol | 0.9 | 0.9 | 0.9 | 0.9 | 0.9 | 0.9 | 0.9 | 0.9 |
| Ethylhexylglycerin | 0.09 | 0.09 | 0.09 | 0.09 | 0.09 | 0.09 | 0.09 | 0.09 |
| Water | QS to 100 | QS to 100 | QS to 100 | QS to 100 | QS to 100 | QS to 100 | QS to 100 | QS to 100 |

Identical swatches of very curly hair were washed with a standard shampoo, rinsed, and detangled (Time -1). Equal amounts of one of inventive compositions 3A-3B or comparative compositions C6-C11 were applied to separate wet hair swatches and distributed by raking through the curls to ensure even distribution (Time 0). The treated hair swatches were then allowed to air dry for 18 hours (Time 1). The treated swatches were then put into a humidity chamber at 80% relative humidity at 25° C. for 5 hours (Time 2). The hair swatches treated with each of compositions 3A-3B or C6-C11 were evaluated for elongation (length retention), curl definition, and frizz at each of Time -1, Time 0, Time 1, and Time 2.

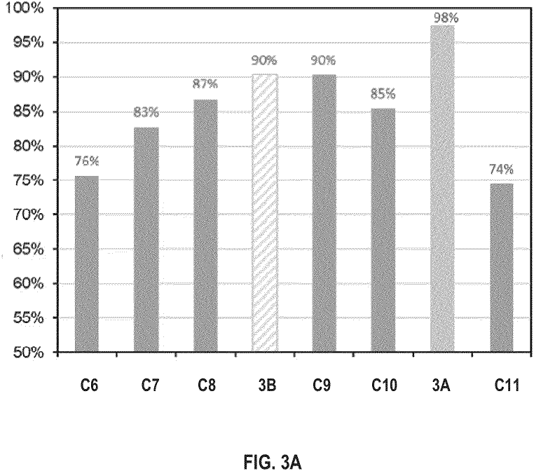

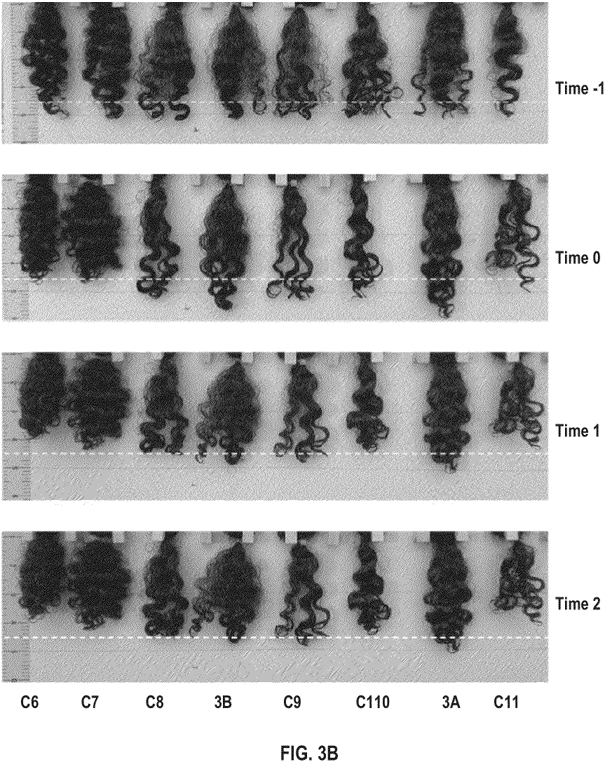

FIG. 3A shows that inventive hair styling composition 3A surprisingly provided improved length retention to very curly hair, compared with comparative hair styling compositions C6-C11. FIG. 3B shows that each of the swatches treated with inventive compositions 3A or 3B demonstrated markedly better elongation, curl definition, and frizz control than the swatches treated with comparative compositions C6-C11 at each time point, even when subjected to high humidity.

Example 3-1 thus demonstrates that hair treatment compositions according to the disclosure surprisingly provide better elongation (length retention), curl definition, and frizz control to curly hair than hair treatment compositions not according to the disclosure.

### Example 3-2 - Hair Styling Compositions

The study in Example 3-1 was repeated a second time on a second set of very curly hair swatches, with similar results wherein inventive compositions 3A-3B provided better elongation (length retention), curl definition, and frizz control to curly hair than comparative compositions C6-C11, even when the hair was subjected to high humidity.

Example 3-2 therefore confirms the results of Example 3-1 that compositions according to the disclosure provide surprising styling benefits to curly hair.

### Example 4 — Compositions With Gluconolactone and Various Amino Acids

The inventive and comparative hair styling compositions in Table 4 were prepared by combining the components shown with water and a pH adjustor (either citric acid or trisodium citrate) in a quantity sufficient to achieve a final pH in the range of about 4.5 to about 5.3.

TABLE 4

| | | Glucono -lactone | Taurine | Arginine | Serine | Threonine | Phenylalanine | Glycine | pH adj. | Water |
|---|---|---|---|---|---|---|---|---|---|---|
| Inventive | 4A | 1 | 2 | | | | | | QS | QS to 100 |
| | 4B | 1 | | 2 | | | | | QS | QS to 100 |
| | 4C | 1 | | | 2 | | | | QS | QS to 100 |
| | 4D | 1 | | | | 2 | | | QS | QS to 100 |
| | 4E | 1 | | | | | 2 | | QS | QS to 100 |
| | 4F | 1 | | | | | | 2 | QS | QS to 100 |
| Comparative | C12 | | 2 | | | | | | QS | QS to 100 |
| | C13 | | | 2 | | | | | QS | QS to 100 |
| | C14 | | | | 2 | | | | QS | QS to 100 |
| | C15 | | | | | 2 | | | QS | QS to 100 |
| | C16 | | | | | | 2 | | QS | QS to 100 |
| | C17 | | | | | | | 2 | QS | QS to 100 |
| | C18 | 1 | | | | | | | QS | QS to 100 |

Identical swatches of very curly hair were washed with a standard shampoo, rinsed, and detangled (Time -1). Equal amounts of one of inventive compositions 4A-4F or comparative compositions C12-C18 were applied to separate wet hair swatches and distributed by raking through the curls to ensure even distribution (Time 0). The treated hair swatches were allowed to air dry for 18 hours (Time 1). The treated swatches were then put into a humidity chamber at 80% relative humidity at 25° C. for 5 hours (Time 2). The hair swatches treated with compositions 4A-4F or C12-C18 were evaluated for elongation (length retention), curl definition, and frizz at each of Time -1, Time 0, Time 1, and Time 2.

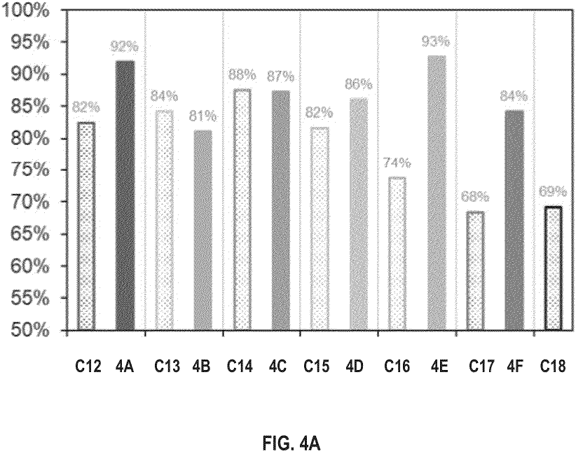

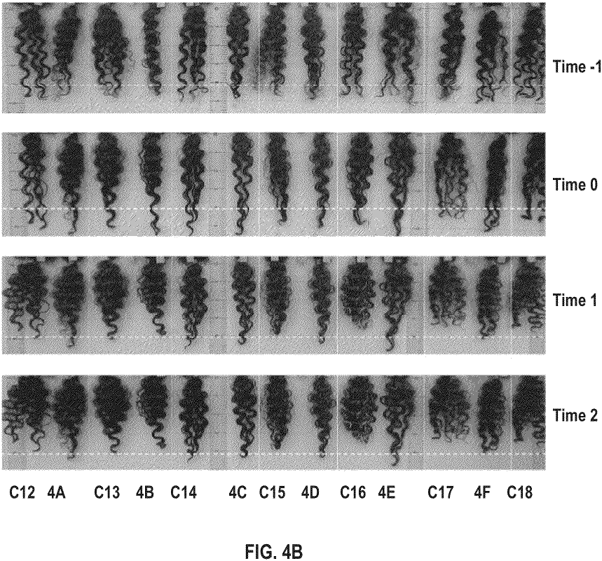

FIG. 4A shows that inventive hair styling compositions 4A-4F surprisingly provided improved length retention to very curly hair, compared with comparative hair styling compositions C12-C18. FIG. 4B shows that each of the swatches treated with inventive compositions 4A-4F demonstrated markedly better elongation, curl definition, and frizz control than the swatches treated with comparative compositions C12-C18 at each time point, even when subjected to high humidity.

Example 4 thus demonstrates that hair styling compositions according to the disclosure having gluconolactone in combination with various amino acids surprisingly provide better elongation (length retention), curl definition, and frizz control to curly hair than hair treatment compositions not according to the disclosure.

The above examples demonstrate that compositions according to the disclosure having gluconolactone and at least one compound chosen from amino acids, amino sulfonic acids, salts thereof, or mixtures thereof provide surprising and unexpected synergistic benefits such as improved curl elongation, lasting curl elongation, and/or frizz control to hair. These results, which are considered to be statistically significant, confirm that compositions comprising a solvent, optionally a polysaccharide, and from about 0.05% to about 10% of amino acids, amino sulfonic acids, and salts thereof would be expected to provide the same synergistic benefits in combination with about 0.05% to about 10% of gluconolactone described herein, under similar conditions.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-36. (canceled)

37. A method for improving the elongation of curly hair, comprising applying to the hair a composition comprising:
(a) gluconolactone,
(b) at least one compound chosen from amino acids, amino sulfonic acids, salts thereof, or mixtures thereof, and
(c) at least one solvent,
wherein the pH of the composition is less than about 7.

38. The method according to claim 37, wherein the total amount of (a) gluconolactone ranges from about 0.05% to about 10% by weight, relative to the total weight of the composition.

39. The method according to claim 37, wherein the total amount of (b) compounds chosen from amino acids, amino sulfonic acids, salts thereof, or mixtures thereof ranges from about 0.05% to about 10% by weight, relative to the total weight of the composition.

40. The method according to claim 37, wherein the composition comprises at least one amino acid or amino sulfonic acid chosen from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, minomethane sulfonic acid, taurine, aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, salts thereof, or mixtures thereof.

41. The method according to claim 37, wherein the composition further comprises at least one anionic polysaccharide.

42. The method according to claim 37, wherein the composition further comprises at least one thickening agent.

43. The method according to claim 37, wherein the pH of the composition ranges from about 3 to about 6.

44. The method according to claim 37, wherein the composition further comprises at least one film forming polymer.

45. The method according to claim 37, wherein the composition further comprises at least one anionic surfactant.

46. The method according to claim 37, wherein the weight ratio of the total amounts of (b) amino acids + amino sulfonic acids + salts:(a) gluconolactone in the composition ranges from about 5:1 to about 1:1.

47. A method for improving the elongation of curly hair, comprising applying to the hair a composition comprising:
(a) from about 0.05% to about 10% of gluconolactone,
(b) from about 0.05% to about 10% of compounds chosen from amino acids, amino sulfonic acids, salts thereof, or mixtures thereof, and
(c) at least one solvent,
wherein the weight ratio of the total amounts of (b) amino acids + amino sulfonic acids + salts:(a) gluconolactone in the composition ranges from about 5:1 to about 1:1; and
wherein all amounts are by weight, relative to the total weight of the composition.

48. The method according to claim 47, wherein the composition comprises at least one amino acid or amino sulfonic acid chosen from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, minomethane sulfonic acid, taurine, aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, salts thereof, or mixtures thereof.

49. The method according to claim 47, wherein the composition comprises at least one compound chosen from phenylalanine, glycine, taurine, salts thereof, or mixtures thereof.

50. The method according to claim 47, wherein the composition further comprises at least one thickening agent.

51. The method according to claim 47, wherein the composition further comprises at least one anionic polysaccharide.

52. The method according to claim 47, wherein the composition further comprises at least one film forming polymer.

53. The method according to claim 47, further comprising at least one anionic surfactant.

54. The method according to claim 47, wherein the pH of the composition is less than about 7.

55. The method according to claim 47, wherein the weight ratio of the total amounts of (b) amino acids + amino sulfonic acids + salts:(a) gluconolactone in the composition ranges from about 4:1 to about 1:1.

56. A method for styling hair, comprising applying to the hair a composition comprising:
(a) from about 0.5% to about 3% of gluconolactone,
(b) from about 1% to about 3% of compounds chosen from phenylalanine, glycine, taurine, salts thereof, or mixtures thereof, and
(c) water,
wherein the weight ratio of the total amount of (b) compounds chosen from phenylalanine, glycine, taurine, and salts thereof:(a) gluconolactone in the composition ranges from about 3:1 to about 1:1,
wherein the pH of the composition ranges from about 3 to about 6, and
wherein all amounts are by weight, relative to the total weight of the composition.
